(19) United States
(12) Patent Application Publication
(10) Pub. No.: US 2001/0056087 A1
(43) Pub. Date: Dec. 27, 2001

(54) Use of 5alpha-androst-1en-3,17-diol to increase the level of the anabolic/androgenic hormone 17beta-hydroxy-5alpha-androst-1-en-3-one in humans

(76) Inventor: Patrick Arnold, Seymour, IL (US)

Correspondence Address: Berns Law Office, P.C., 107 W. Goose Alley, Urbana, IL, 61801, US

(21) Appl. No.: 09/756,519

(22) Filed: Jan. 9, 2001

Related U.S. Application Data
(63) Continuation-in-part of application No. 09/603,097, filed on Jun. 26, 2000, now abandoned.

(51) Int. Cl. A61K 31/56

(52) U.S. Cl. 514/182

(57) ABSTRACT

The invention involves the administration of the natural androgen metabolite 5alpha-androst-1-en-3,17-diol (1-androstenediol) to produce an androgenic and anabolic response in humans. 1-androstenediol behaves as a prohormone in vivo, and converts to the active hormone 17beta-hydroxy-5alpha-androst-1-en-3-one.

### FIELD OF INVENTION

The invention relates to a method of administering 5alpha-androst-1-en-3,17-diol (1-androstenediol), a precursor to the steroid hormone 17beta-hydroxy-5alpha-androst-1-en-3-one. The administration of this precursor will increase the levels of this hormone in humans. The hormone is very similar to testosterone, only it is much more potent for maintenance of muscle and bone mass.

### DESCRIPTION OF THE PRIOR ART

U.S. Pat. No. 5,880,117 to Arnold discloses a method of effectively increasing testosterone levels in humans by the administration of the testosterone precursor 4-androstenediol. Similarly, U.S. Pat. No. 6,011,027 to Arnold discloses a method for increasing nortestosterone levels in humans by administration of the nortestosterone metabolic precursor 19-nor-4-andrsotenediol. U.S. Pat. No. 5,578,588 to Mattern et al. teaches the usage of 4-androstenedione as a metabolic precursor to increase levels of testosterone in humans, The pharmacokinetics of the oral administration of such metabolic precursors is such that a peak in blood levels is seen at approximately 90 minutes with a subsequent decline to baseline within 3-4 hours. This fact permits one to more closely simulate the natural endogenous pulsatile release of testosterone through multiple daily dosing of androgenic precursors. This should result in a more normal physiological response with a minimization of side effects and HPTA shutdown. Furthermore, since these precursors are not 17alpha alkylated compounds, their hepatotoxicity is minimal.

### DETAILED DESCRIPTION OF THE PREFERRED EMBODIMENT

A co-pending U.S. patent application of the present inventor introduced the prohormone 5alpha-androst-1-en-3,17-dione (1-androstenedione). 1-androstenedione has been shown to be present as a urinary androgen metabolite in both healthy and diseased men (J. Biol. Chem. (182), 299). This compound can interact in-vivo with the enzyme 17beta-hydroxysteroid dehydrogenase to form the active anabolic/androgenic hormone 17beta-hydroxy-5alpha-androst-1-en-3-one. Evidence for this in-vivo conversion exists in the literature. One hundred mg of 1-androstenedione given orally to men was found to result in considerable excretion of 17beta-hydroxy-5alpha-androst-1-en-3-one in the urine (J. Steroid Biochem., (3) 933).

5-alpha-androst-1-en-3,17-diol (1-androstenediol) is a product manufactured by the hydride reduction of 1-androstenedione. The product thus obtained consists predominantly of the 3beta,17beta isomer. 1-androstenediol has the ability to convert in-vivo to 17beta-hydroxy-5alpha-androst-1-en-3-one through its interaction with the endogenous enzyme 3beta-hydroxysteroid dehydrogenase, in the same way as 4-androstenediol converts to testosterone. Therefore, like 1-androstenedione, 1-androstenediol behaves as a prohormone for 17beta-hydroxy-5alpha-androst-1-en-3-one when taken into the body.

As is also the case with 1-androstenedione, 1-androstenediol maintains a unique advantage over other prohormones with 4(5) unsaturation. It is metabolized differently than these other prohormones as it relates to the ultimate degree of formation of 17-keto steroid metabolites. The enzyme 17beta-hydroxysteroid dehydrogenase catalyses the reversible transformation of the biologically active 17beta-hydroxysteroids to the biologically inactive 17-keto steroids. This reaction is an equilibrium, and for 1(2) unsaturated steroids this equilibrium lies considerably more towards the formation of active 17beta-hydroxysteroids than it does for 4(5) unsaturated steroids (Acta Endocrinologica, (41) 494). As a result, the administration of 1-androstenediol will result in the overall formation of the more active 17beta-hydroxyl hormone in the body then 4(5) unsaturated prohormones such as 4-androstenedione, 4-androstenediol, and 19-nor-4-androstenediol will.

Therefore, 1-androstenediol can be used as a superior androgen metabolic precursor to raise levels of 17beta-hydroxy-5alpha-androst-1-en-3-one and impart an anabolic/androgenic response in humans.

Oral 1-androstenediol can be given in an effective amount, daily doses of 25 mg to 2000 mg; preferably 100 mg to 1000 mg. These daily doses can be divided into several subdoses with 2-4 being most preferable. In addition to peroral administration, 1-androstenediol can also be effectively administered by several other routes including transdermal, rectal (suppository), intranasal, and sublingual. A particular advantageous method of sublingual administration involves complexing 1-androstenediol with beta-hydroxypropyl-beta-cyclodextrin, which is then pressed into tablets.

I claim:

1. A method of increasing the 17beta-hydroxy-5alpha-androst-1-en-3-one levels in humans by the administration of an effective amount of 1-androstenediol.

2. The method of claim 1 wherein said administration is peroral.

3. The method of claim 1 wherein said administration is selected from the group consisting of transdermal, rectal, intranasal, and sublingual.

4. The method of claim 1, wherein said effective amount is a daily dosage of 25 to 2000 mg.

5. A method of imparting an androgenic/anabolic effect in humans by the administration of an effective amount of 1-androstenediol.

6. The method of claim 5 wherein said administration is peroral.

7. The method of claim 5 wherein said administration is selected from the group consisting of transdermal, rectal, intranasal, and sublingual.

8. The method of claim 5, wherein said effective amount is a daily dosage of 25 to 2000 mg.
